(19) United States
(12) Patent Application Publication
(10) Pub. No.: US 2024/0307641 A1
(43) Pub. Date: Sep. 19, 2024

(54) CPAP SYSTEM, CPAP DEVICE, AND CPAP DEVICE PROGRAM

(71) Applicant: Murata Manufacturing Co., Ltd., Kyoto (JP)

(72) Inventors: Masayuki SHIRAI, Kyoto (JP); Takanori HAYASHI, Kyoto (JP); Hiroki JINNOUCHI, Kyoto (JP)

(21) Appl. No.: 18/603,423

(22) Filed: Mar. 13, 2024

(30) Foreign Application Priority Data
Mar. 17, 2023 (JP) 2023-043154

(51) Int. Cl. A61M 16/00; G16H 40/63

(52) U.S. Cl. CPC A61M 16/024; A61M 16/0066; G16H 40/63; A61M 2205/3553; A61M 2205/50; A61M 2205/52

(57) ABSTRACT

A CPAP system includes a CPAP device and a reception part. The CPAP device is capable of executing a determination process, a first transmission process, and a second transmission process. The determination process is a process in which a determination about whether a respiratory therapy with the CPAP device has been performed within a predetermined fixed period is made. The first transmission process is a process in which execution completion information is transmitted to the reception part in response to a determination in the determination process. The second transmission process is a process in which non-execution information is transmitted to the reception part in response to a determination in the determination process. The reception part is capable of executing a surmise process in which a non-therapy period in which the respiratory therapy has not been performed is surmised based on the execution completion information and the non-execution information.

### CROSS REFERENCE TO RELATED APPLICATION

This application claims priority from Japanese Patent Application No. 2023-043154 filed on Mar. 17, 2023. The content of this application is incorporated herein by reference in its entirety.

### BACKGROUND OF THE DISCLOSURE

### 1. Field of the Disclosure

The present disclosure relates to a CPAP system, a CPAP device, and a CPAP device program.

### 2. Description of the Related Art

A respiratory pressure therapy device described in Japanese Unexamined Patent Application Publication No. 2019-217288 stores therapy data in the memory. The therapy data includes data such as a respiratory flow rate acquired during the therapy session. The respiratory pressure therapy device is capable of communicating with a server. The respiratory pressure therapy device transmits the therapy data stored in the memory to the server. Based on the therapy data, the server computes an analysis result such as an average mask pressure in the therapy session period.

### BRIEF SUMMARY OF THE DISCLOSURE

The respiratory pressure therapy device as described in Japanese Unexamined Patent Application Publication No. 2019-217288 is desirably used continuously at bedtime every day. In the respiratory pressure therapy device described in Japanese Unexamined Patent Application Publication No. 2019-217288, transmitting the therapy data to the server enables the server to detect the implementation of the therapy. However, if the therapy data is not transmitted from the respiratory pressure therapy device, it is difficult for the server to correctly determine whether the therapy is executed.

To address the issue above, the present disclosure provides a CPAP system including: a CPAP device; and a reception part configured to communicate with the CPAP device. The CPAP device is configured to execute a determination process in which a determination about whether a respiratory therapy with the CPAP device has been performed within a predetermined fixed period is made, a first transmission process in which execution completion information is transmitted to the reception part in response to a determination in the determination process, the determination indicating that the respiratory therapy has been performed within the fixed period, the execution completion information indicating that the respiratory therapy has been performed within the fixed period, a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period. The reception part is configured to execute a surmise process in which a non-therapy period in which the respiratory therapy has not been performed is surmised based on the execution completion information and the non-execution information.

To address the issue above, the present disclosure provides a CPAP device configured to execute: a determination process in which a determination about whether a respiratory therapy has been performed within a predetermined fixed period is made; a first transmission process in which execution completion information is transmitted to the reception part, the execution completion information indicating that the respiratory therapy has been performed within the fixed period, the execution completion information being transmitted in response to a determination in the determination process, the determination indicating that the respiratory therapy has been performed within the fixed period, and a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period.

To address the issue above, the present disclosure provides a CPAP device program that is applied to a CPAP device including a control device, the CPAP device program causing the control device to execute: a determination process in which a determination about whether a respiratory therapy has been performed within a predetermined fixed period is made, a first transmission process in which execution completion information is transmitted to a reception part in response to a determination in the determination process, the determination indicating that the respiratory therapy has been performed within the fixed period, the execution completion information indicating that the respiratory therapy has been performed within the fixed period, and a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period.

According to the configuration above, at the time point when the non-execution information transmitted by the CPAP device in the second transmission process is received by the reception part, the reception part may correctly surmise that the respiratory therapy has not been performed within the fixed period.

The reception part may surmise whether the respiratory therapy is executed in the CPAP device.

### BRIEF DESCRIPTION OF THE SEVERAL VIEWS OF THE DRAWINGS

FIG. 1 is a schematic configuration view of a CPAP system;

FIG. 2 is a perspective view of the CPAP system;

FIG. 3 is a schematic configuration view of a CPAP device;

FIG. 4 is a flowchart illustrating a series of steps in information transmission control performed by a control device of the CPAP device;

FIG. 5 is a flowchart illustrating a series of steps in in-communication-interruption control performed by the control device of the CPAP device; and

FIG. 6 is a sequence diagram for explaining an example process by the CPAP device.

### DETAILED DESCRIPTION OF THE DISCLOSURE

Hereinafter, an embodiment of a CPAP system, a CPAP device, and a CPAP device program will be described with reference to the drawings. The drawings illustrate enlarged components on occasions for easy understanding. The ratio of the dimensions of the components are different from an actual ratio or a ratio in a different drawing on occasions.

### CPAP System

As illustrated in FIG. 1, a CPAP system 500 includes a CPAP device 10, a server 200, and a display 300.

The CPAP device 10 includes a control device 100. The control device 100 may be configured as circuitry including one or more processors that execute various processes in accordance with a computer program (software). The control device 100 may be configured as one or more dedicated hardware circuits, such as an application specific integrated circuit (ASIC), that execute at least part of the various processes, or the combination thereof. The processor includes a central processing unit (CPU) and a memory such as a random-access memory (RAM) and a read-only memory (ROM). The memory stores program code or instructions configured to cause the CPU to execute a process. The memory, that is, a computer readable medium includes any usable medium accessible by a general-purpose or dedicated computer.

The control device 100 is capable of communicating with the server 200 via a wireless communication network (not illustrated). The server 200 is an example of a reception part. Although the illustration of the server 200 is omitted, the server 200 has a CPU and a ROM. The CPU of the server 200 runs a program stored in the ROM. The server 200 is thereby capable of transmitting a signal to the display 300. The display 300 is an example of an external device different from the CPAP device 10.

### CPAP Device Structure

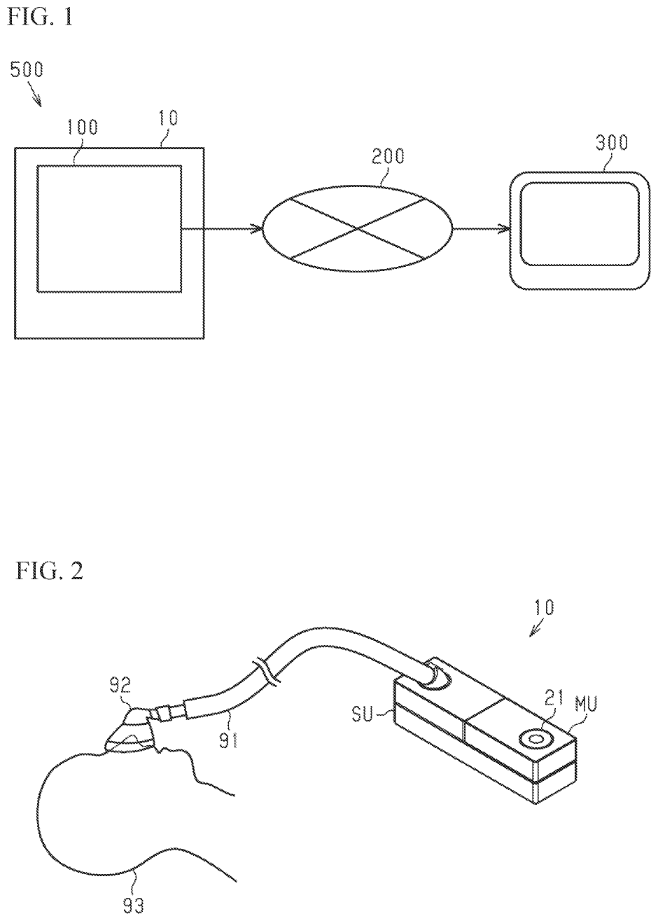

The overall configuration of the CPAP device 10 will be described. As illustrated in FIG. 2, the CPAP device 10 includes a main unit MU and a sub unit SU. In a state where the sub unit SU is attached to the main unit MU, that is, in an in-use state of the CPAP device 10, the CPAP device 10 is in the shape of a substantially rectangular parallelepiped as a whole.

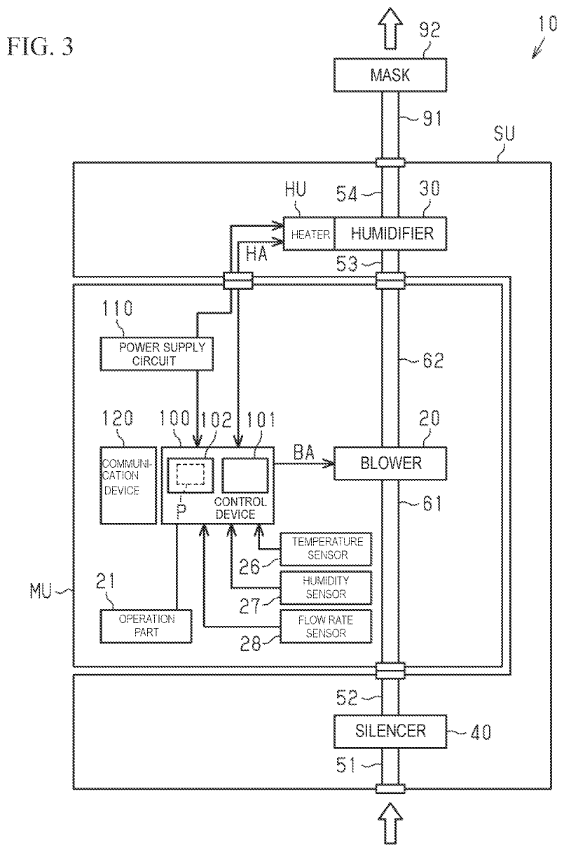

The main unit MU of the CPAP device 10 is in the shape of a substantially rectangular parallelepiped. As illustrated in FIG. 3, the main unit MU has a blower 20. The blower 20 accommodates an air blow fan for pressure-feeding air. The main unit MU also has an upstream passage 61 and a downstream passage 62. The upstream passage 61 and the downstream passage 62 are defined in the main unit MU. The upstream passage 61 and the downstream passage 62 are connected to the blower 20. The blower 20 sucks up air in the upstream passage 61 and pressure-feeds the air to the downstream passage 62.

As illustrated in FIG. 2, the main unit MU has an operation part 21. The operation part 21 is located on the upper surface of the main unit MU. The operation part 21 is provided for operations such as turning on and off the blower 20.

The sub unit SU of the CPAP device 10 is in an L-shape in a side view in a width direction. By fitting the main unit MU in the inside of the L-shape of the sub unit SU, the CPAP device 10 forms the substantially rectangular parallelepiped as described above.

As illustrated in FIG. 3, the sub unit SU has a humidifier 30, a silencer 40, and a heater unit HU. The silencer 40 is provided to silence a noise generated in response to driving the blower 20. Although the illustration of the silencer 40 is omitted, the silencer 40 has a flow passage for the air and a silencer chamber to which the variation in the pressure of the air in the flow passage is transmittable. The humidifier 30 is capable of storing water therein. The air fed from the air blow fan of the blower 20 under pressure may flow into the humidifier 30.

The heater unit HU is adjacent to the humidifier 30. The heater unit HU has a heater resistor as a heating element. The heater unit HU heats the water stored in the humidifier 30. The water in the humidifier 30 is heated by the heater unit HU and thereby volatilizes easily. The air in the humidifier 30 is thereby humidified.

The sub unit SU has a first passage 51, a second passage 52, a third passage 53, and a fourth passage 54. The first passage 51 is defined in the sub unit SU. The first end of the first passage 51 is open to the outside of the sub unit SU. The second end of the first passage 51 is connected to the silencer 40. The second passage 52 is defined in the sub unit SU. The first end of the second passage 52 is connected to the silencer 40. The second end of the second passage 52 is open to the outside of the sub unit SU.

The third passage 53 is defined in the sub unit SU. The first end of the third passage 53 is open to the outside of the sub unit SU. The second end of the third passage 53 is connected to the humidifier 30. The fourth passage 54 is defined in the sub unit SU. The first end of the fourth passage 54 is connected to the humidifier 30. The second end of the fourth passage 54 is open to the outside of the sub unit SU.

As illustrated in FIG. 2, the CPAP device 10 includes a hose 91 and a mask 92. As illustrated in FIG. 3, the first end of the hose 91 is connected to the second end of the fourth passage 54. The mask 92 is connected to the second end of the hose 91. As illustrated in FIG. 2, the mask 92 is worn by a user 93 in such a manner as to cover the nose or the mouth of the user 93. Although the illustration of the hose 91 is omitted, the first end of the hose 91 may be connected to the downstream passage 62 in the main unit MU.

### Air Flowing Path in In-Use State

As described above, the sub unit SU is attachable to the main unit MU. In the state where the sub unit SU is attached to the main unit MU, the passages in the main unit MU and the passages in the sub unit SU become interconnected. Specifically, as illustrated in FIG. 3, the upstream passage 61 in the main unit MU is connected to the second end of the second passage 52 in the sub unit SU. The downstream passage 62 in the main unit MU is also connected to the first end of the third passage 53 in the sub unit SU. Accordingly, in response to the blower 20 being driven, the air taken from the first passage 51 reaches the blower 20 via the silencer 40, the second passage 52, and the upstream passage 61. The air fed by the blower 20 under pressure then reaches the mask 92 via the downstream passage 62, the third passage 53, the humidifier 30, the fourth passage 54, and the hose 91.

Although the illustration of the CPAP device 10 is omitted, the CPAP device 10 is also usable in a state where the sub unit SU is detached from the main unit MU. In this case, the air reaches the mask 92 via the upstream passage 61, the blower 20, the downstream passage 62, and the hose 91.

### Electrical Configuration of CPAP Device

As illustrated in FIG. 3, the main unit MU includes a temperature sensor 26, a humidity sensor 27, and a flow rate sensor 28. The temperature sensor 26 detects the temperature of the air in the upstream passage 61. The humidity sensor 27 detects the relative humidity of the air in the upstream passage 61. The flow rate sensor 28 detects a flow rate per unit time of air flowing through the upstream passage 61.

The main unit MU includes the control device 100, a power supply circuit 110, and a communication device 120. The control device 100 has a function of communicating with the server 200 described above and is also capable of executing the control of the components of the CPAP device 10.

The control device 100 includes a memory 102 and a computing unit 101. The computing unit 101 is capable of running various programs stored in the memory 102. The computing unit 101 is capable of writing data to the memory 102 in running a program. The memory 102 includes a ROM that is only readable, a readable and writable volatile RAM, and a readable and writable nonvolatile storage. The memory 102 also stores a CPAP device program P for performing information transmission control and in-communication-interruption control that are to be described later.

The control device 100 acquires, from the operation part 21, a signal indicating an operation performed of the operation part 21. Based on the signal acquired from the operation part 21, the control device 100 outputs a control signal BA to the blower 20. The control device 100 controls the turning on and off, the number of revolutions, and the like of the blower 20 by using the control signal BA. The control device 100 also outputs a control signal HA to the heater unit HU. The control device 100 controls the heating value of the heater unit HU by using the control signal HA.

The control device 100 is also capable of executing a storing process in which therapy data during the respiratory therapy execution by the CPAP device 10 is acquired. Specifically, the control device 100 chronologically acquires data indicating the temperature detected by the temperature sensor 26 during the respiratory therapy execution by the CPAP device 10. The control device 100 also chronologically acquires data indicating the relative humidity detected by the humidity sensor 27 during the respiratory therapy execution by the CPAP device 10. Further, the control device 100 chronologically acquires data indicating the flow rate detected by the flow rate sensor 28 during the respiratory therapy execution by the CPAP device 10. The control device 100 stores the pieces of chronological data as collective therapy data.

The power supply circuit 110 receives direct-current (DC) power from an external apparatus (not illustrated). The power supply circuit 110 supplies the DC power from the external apparatus to electrical devices such as the control device 100 and the blower 20. In the state where the sub unit SU is attached to the main unit MU, the power supply circuit 110 supplies the power to the heater unit HU of the sub unit SU. Although the illustration of the communication device 120 is omitted, the communication device 120 has an antenna that transmits a radio wave and a peripheral circuit thereof. The communication device 120 performs communication in accordance with, for example, the IEEE 802 11 standard.

### Information Transmission Control by Control Device

The computing unit 101 of the control device 100 runs the CPAP device program P stored in the memory 102 and thereby performs steps in the information transmission control. Hereinafter, “executing a process by the computing unit 101 based on the CPAP device program P stored in the memory 102” is simply referred to as executing a process by the control device 100 on occasions.

After the state of the power supply of the CPAP device 10 is changed from an off state to an on state, the control device 100 performs the information transmission control after the elapse of a predetermined fixed period. The control device 100 then performs the information transmission control every fixed period described above until the power supply of the CPAP device 10 enters the off state. An example of the fixed period is 24 hours.

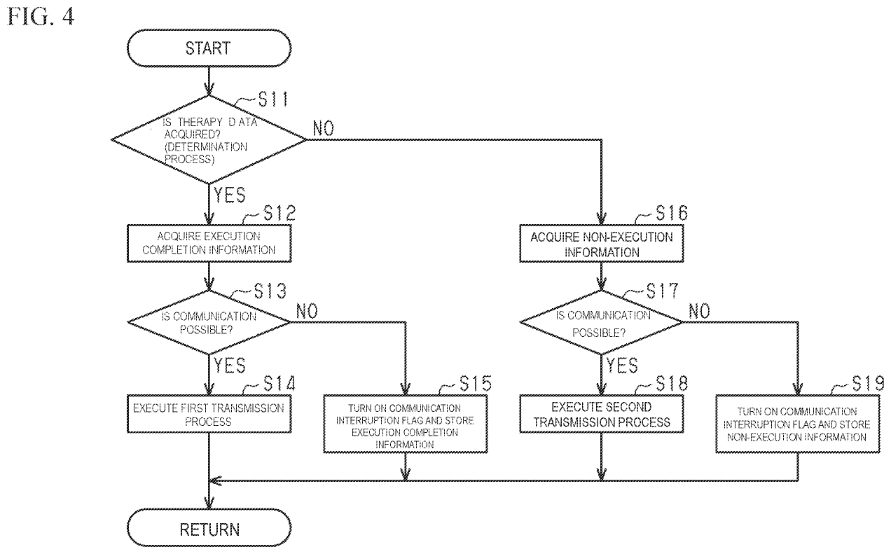

As illustrated in FIG. 4, after starting the information transmission control, the control device 100 first executes a determination process in step S11. Specifically, in step S11, the control device 100 determines whether the therapy data has been acquired within a past fixed period from the time point of the process in step S11. The process in step S11 is thus a determination process in which whether the respiratory therapy with the CPAP device 10 has been performed within the fixed period is determined. If the determination result is affirmative in step S11, the control device 100 performs step S12.

In step S12, the control device 100 acquires execution completion information indicating that the respiratory therapy with the CPAP device 10 has been performed within the fixed period. Thereafter, the control device 100 performs step S13.

In step S13, the control device 100 determines whether the control device 100 is in a state where communication with the server 200 is possible. If the communication with the server 200 is possible, that is, if the determination result is affirmative in step S13, the control device 100 performs step S14.

In step S14, the control device 100 executes a first transmission process. Specifically, the control device 100 transmits the execution completion information to the server 200 by using the communication device 120. In the first transmission process, the control device 100 also transmits, to the server 200, data regarding the therapy in the period in which the respiratory therapy has been performed and that is indicated in the execution completion information, in addition to the execution completion information. At this time, the control device 100 transmits the execution completion information in relation to the therapy data. Thereafter, the control device 100 terminates the series of steps in the information transmission control. The control device 100 performs the information transmission control again after the elapse of the fixed period.

If the determination result is negative in step S13, the control device 100 performs step S15. In step S15, the control device 100 turns on a communication interruption flag indicating that the communication with the server 200 is interrupted. The control device 100 also stores the execution completion information acquired in step S12 in relation to the corresponding therapy data. Thereafter, the control device 100 terminates the series of steps in the information transmission control. The control device 100 performs the information transmission control again after the elapse of the fixed period.

In contrast, if the determination result is negative in step S11, the control device 100 performs step S16. In step S16, the control device 100 acquires non-execution information indicating that the respiratory therapy with the CPAP device 10 has not been performed within the fixed period. Thereafter, the control device 100 performs step S17.

In step S17, the control device 100 determines whether the control device 100 is in the state where the communication with the server 200 is possible. If the communication with the server 200 is possible, that is, if the determination result is affirmative in step S17, the control device 100 performs step S18.

In step S18, the control device 100 executes a second transmission process. Specifically, the control device 100 transmits the non-execution information to the server 200 by using the communication device 120. In the case of step S18, the respiratory therapy has not been performed, and the corresponding therapy data is absent. Accordingly, in step S18, the control device 100 transmits only the non-execution information to the server 200. Thereafter, the control device 100 terminates the series of steps in the information transmission control. The control device 100 performs the information transmission control again after the elapse of the fixed period.

If the determination result is negative in step S17, the control device 100 performs step S19. In step S19, the control device 100 turns on the communication interruption flag indicating that the communication with the server 200 is interrupted. The control device 100 also stores the non-execution information acquired in step S16. Thereafter, the control device 100 terminates the series of steps in the information transmission control. The control device 100 performs the information transmission control again after the elapse of the fixed period.

As described above, the control device 100 of the CPAP device 10 repeatedly executes the first transmission process or the second transmission process in a predetermined specified cycle. In this embodiment, the specified cycle is a cycle period periodically segmented every fixed period described above. The specified cycle is thus a 24-hour cycle.

### In-Communication-Interruption Control by Control Device

The computing unit 101 of the control device 100 runs the CPAP device program P stored in the memory 102 and thereby performs steps in the in-communication-interruption control. On condition that the communication interruption flag is turned on in the information transmission control, the control device 100 performs the in-communication-interruption control only one time. The control device 100 is capable of performing the in-communication-interruption control concurrently with the information transmission control described above.

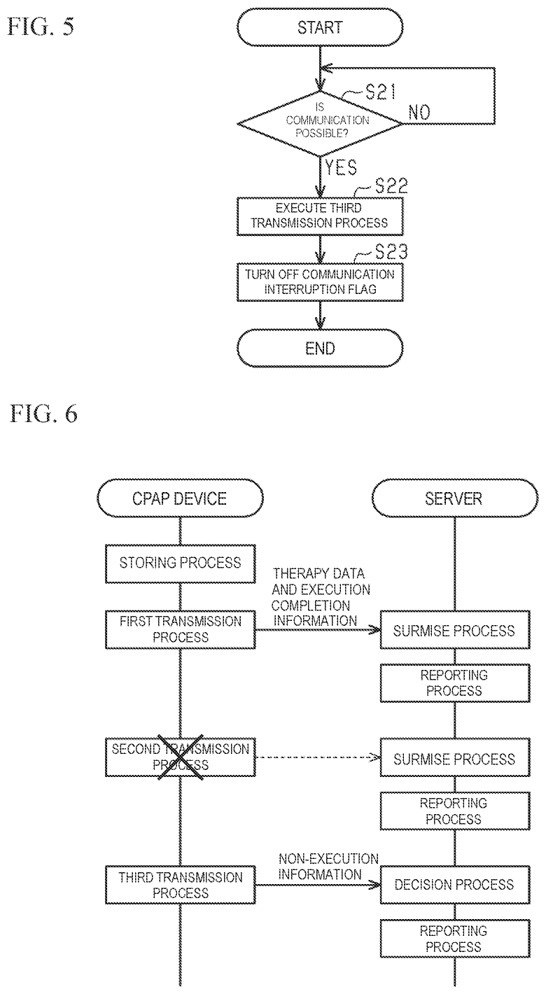

As illustrated in FIG. 5, after starting the in-communication-interruption control, the control device 100 first performs step S21. In step S21, the control device 100 determines whether the control device 100 is in the state where the communication with the server 200 is possible. If the determination result is negative in step S21, the control device 100 again performs step S21. That is, the control device 100 repeatedly performs step S21 until the communication with the server 200 becomes possible. In contrast, if the determination result is affirmative in step S21, the control device 100 performs step S22.

In step S22, the control device 100 executes a third transmission process. Specifically, the control device 100 transmits, to the server 200, the execution completion information and the non-execution information that are stored during the information transmission control and have not been transmitted to the server 200 at the time point of step S22.

As described above, if the communication with the server 200 is interrupted, the control device 100 holds the transmission of the execution information and the non-execution information. Specifically, if the communication with the server 200 is interrupted when the execution of the first transmission process or the second transmission process is tried during the information transmission control, the server 200 holds the transmission of the execution information and the non-execution information. After the communication with the server 200 becomes possible, the control device 100 transmits the execution completion information and the non-execution information that are stored, to the server 200 in the in-communication-interruption control. In succeeding step S23, the control device 100 turns off the communication interruption flag that has turned on in the information transmission control. The control device 100 terminates the series of steps in the in-communication-interruption control.

### Control by Server

The server 200 is capable of executing the surmise process, a reporting process, and a decision process. When receiving the execution completion information or the non-execution information from the CPAP device 10, the server 200 executes the surmise process. Specifically, when receiving the execution completion information from the CPAP device 10, the server 200 surmises, in the surmise process, that the respiratory therapy has been performed within the past fixed period. Likewise, when receiving the non-execution information from the CPAP device 10, the server 200 surmises that the respiratory therapy has not been performed within the past fixed period. The server 200 repeats the steps as described above, and thereby surmises a non-therapy period indicating on which day within a target period, such as one week or one month, the respiratory therapy is performed.

If the receiving of both of the execution completion information and the non-execution information within a given period fails, the server 200 identifies the given period as a blank period. In this embodiment, the given period corresponds to a period in the specified cycle. In other words, if the receiving of both of the execution completion information and the non-execution information within 24 hours fails, the server 200 identifies the period in the specified cycle as the blank period. The server 200 does not make a decision for the blank period regarding whether the respiratory therapy has been performed. The phrase “not make a decision” is referred to for a state where whether the respiratory therapy is performed is not surmisable.

When receiving the execution completion information or the non-execution information in the third transmission process by the CPAP device 10, the server 200 is able to execute the decision process. In other words, when receiving the execution completion information or the non-execution information related to the blank period, the server 200 is able to execute the decision process. In the decision process, the server 200 surmises whether the respiratory therapy has been performed or has not been performed in the blank period in the same manner as in the surmise process described above. That is, the server 200 decides the information indicating whether the respiratory therapy has been performed in the blank period. The server 200 decides the undecided information and thereby surmises a new non-therapy period in a state without the blank period.

After executing the surmise process or the decision process, the server 200 executes the reporting process. In the reporting process, the server 200 outputs the surmised information regarding the non-therapy period to the display 300. Based on the information regarding the non-therapy period, the display 300 displays a message report, for example, “the Yth of Month X: respiratory therapy yet to be implemented; the Zth of Month X: respiratory therapy implemented”. If the blank period is present as the result of the surmise process, the server 200 displays a message report regarding the blank period, for example, “the Bth of Month A: decision not made due to communication interruption”.

### Actions of This Embodiment

Example processes executed by the CPAP device 10 and the server 200 will be described with reference to FIG. 6. First, a user performs the respiratory therapy by using the CPAP device 10 at a certain time point. At this time, in response to the respiratory therapy being performed, the control device 100 executes the storing process. The control device 100 thus stores therapy data during the respiratory therapy.

Thereafter, the specified cycle has elapsed since the previous first or second transmission process. In this example, the respiratory therapy has been performed within the fixed period as described above. The control device 100 thus executes the first transmission process and transmits the execution completion information to the server 200. At this time, the control device 100 transmits the therapy data together with the execution completion information.

Executing the first transmission process by the CPAP device 10 causes the server 200 to acquire the execution completion information. The server 200 then executes the surmise process and the reporting process. Next, the respiratory therapy has not been performed within the fixed period after the first transmission process described above. Further, after the first transmission process, the communication between the CPAP device 10 and the server 200 is interrupted for a period of time longer than the specified period. In this case, the control device 100 tries the second transmission process at a time point when the interruption period exceeds the specified period after the first transmission process. However, it is not possible for the control device 100 to complete the second transmission process because the communication is interrupted.

In contrast, since the CPAP device 10 does not complete the second transmission process, it is possible for the server 200 to receive neither the execution completion information nor the non-execution information. In this case, the server 200 executes the surmise process. At this time in the surmise process, the server 200 does not make a decision for the blank period regarding whether the respiratory therapy has been performed, the blank period being the period in the specified cycle. The server 200 then executes the reporting process.

After the state where completion of the second transmission process fails due to the communication interruption as described above, the CPAP device 10 becomes able to communicate with the server 200. At this time, the control device 100 of the CPAP device 10 executes the third transmission process. The control device 100 thus transmits the non-execution information to the server 200 in the third transmission process.

Based on the non-execution information transmitted in the third transmission process, the server 200 decides that the respiratory therapy has not been performed in the blank period. Based on the execution completion information and the non-execution information that are transmitted in the third transmission process, the server 200 thus executes the decision process in which a decision of whether the respiratory therapy has been performed in the blank period is made. Thereafter, the server 200 executes the reporting process.

### EFFECTS OF THIS EMBODIMENT

(1) In the embodiment above, the control device 100 of the CPAP device 10 is capable of executing the determination process, the first transmission process, and the second transmission process. The server 200 is capable of executing the surmise process. According to this configuration, at the time point when the non-implementation information transmitted by the CPAP device 10 in the second transmission process is received by the server 200, the server 200 may correctly surmise that the respiratory therapy has not been performed within the fixed period.

(2) In the embodiment above, it suffices that the execution completion information is information at least indicating the respiratory therapy has been performed. It also suffices that the non-execution information is information indicating that the respiratory therapy has not been performed. As described above, the execution completion information and the non-execution information may be handled as small size data. According to the embodiment above, the control device 100 may reduce traffic increase in the first transmission process and the second transmission process.

(3) In the embodiment above, if the respiratory therapy is performed, the control device 100 of the CPAP device 10 may execute the storing process in which the therapy data during the respiratory therapy is stored. In the first transmission process, the control device 100 transmits the therapy data to the server 200 in addition to the execution completion information. According to this configuration, the server 200 may collect the therapy data during the respiratory therapy. Accordingly, for example, the medical worker or the like accesses the therapy data stored in the server 200 and thereby may utilize the therapy data for later therapy planning or the like.

(4) In the embodiment above, the server 200 is capable of executing the reporting process in which the information regarding the non-therapy period surmised in the surmise process is reported to the display 300. According to this configuration, for example, a medical worker using the display 300 may comprehend that a patient does not perform the respiratory therapy according to the plan. The medical worker or the like may thus guide the patient based on the information regarding the non-therapy period.

(5) In the embodiment above, if the receiving of both of the in-given-period implementation completion information and the non-implementation information fails in the server 200 in the surmise process, that is, if the blank period is present, the server 200 does not make a decision for the blank period regarding whether the respiratory therapy has been performed. A possibility of wrongly surmising the transmission failure period as the period in which the respiratory therapy has not been performed may thus be prevented, the transmission failure period being a period in which the respiratory therapy has been performed but the transmission of the implementation completion information fails because the communication is interrupted.

(6) In the embodiment above, if the communication with the server 200 is interrupted, the control device 100 is able to execute the third transmission process after the communication with the server 200 becomes possible. The server 200 is also able to execute the decision process in which a decision of whether the respiratory therapy has been performed in the blank period is made, based on the implementation completion information and the non-implementation information that are transmitted in the third transmission process. According to this configuration, whether the respiratory therapy has been performed is decided for the blank period retroactively after the communication with the server 200 becomes possible. It is thus possible to prevent, from continuing for a long time, the blank period that is a period during which a decision regarding whether the respiratory therapy has been performed is not made.

### MODIFICATIONS

The embodiment above and the following modifications may be implemented in combination with each other without technical inconsistency.

In the embodiment above, any structure, for example, any shape of the main unit MU and the sub unit SU may be employed. The CPAP device 10 may also be composed of one unit integrally formed from the main unit MU and the sub unit SU.

In the embodiment above, the server 200 is an example of the reception part. It thus suffices that the reception part is a device capable of communicating with the CPAP device 10. In the embodiment above, the control device 100 may omit the storing process. Executing at least the first transmission process and the second transmission process enables the server 200 to execute the surmise process.

In the embodiment above, the control device 100 may use, as the condition for the affirmative determination in the determination process, a condition different from that in the example in the embodiment above. For example, on condition that the power supply of the CPAP device 10 enters the on state within the fixed period, the control device 100 may determine that the respiratory therapy with the CPAP device 10 has been performed within the fixed period.

In the embodiment above, the control device 100 may transmit the therapy data as the execution completion information to the server 200 in the first transmission process. As long as the therapy data is acquired only when the respiratory therapy is performed, it is possible to handle the therapy data as the information indicating that the respiratory therapy has been performed.

In the embodiment above, any information regarding the non-therapy period may be used as long as the information makes it possible to comprehend a period in which the respiratory therapy has not been performed. For example, the information may be information directly indicating the period in which the respiratory therapy has not been performed and information indirectly indicating the period in which the respiratory therapy has not been performed, the period being indirectly indicated by indicating the period in which the respiratory therapy has been performed and the period the decision for which is not made. In addition, part of the therapy data, a result of a statistic therapy data analysis, and other data may be attached to the information regarding the non-therapy period.

In the embodiment above, the external device different from the CPAP device 10 is not limited to the display 300. For example, the external device may be a smartphone held by the user, the medical worker, and the like. The external device is preferably a device capable of displaying a result of the surmise result in the surmise process executed by the server 200.

In the embodiment above, the server 200 may omit the reporting process. In this case, it suffices that, for example, if a request for outputting the result of the surmise in the surmise process in response to communication connection from an external apparatus, the server 200 outputs the result of the surmise in the surmise process. In addition, in the embodiment above, the CPAP system 500 may not include the display 300.

In the embodiment above, the given period may not coincide with the period in the specified cycle. The given period may be a period predetermined by a user of the server 200. In the embodiment above, the specified cycle may be different from the cycle periodically segmented every fixed period. If the specified cycle is longer than the cycle every fixed period, it is possible for the CPAP device 10 to acquire the execution completion information or the non-execution information in the fixed period.

In the embodiment above, the server 200 may not execute the surmise process in which the decision for the blank period regarding whether the respiratory therapy has been performed is not made. In this case, it is possible for the medical worker using the display 300 to surmise the presence of the blank period during which the decision regarding the presence or absence of the respiratory therapy is not made because the indication of the execution completion information and the non-execution information is not verified.

In the embodiment above, the control device 100 may perform the in-communication-interruption control at timing different from that in the embodiment above. For example, the control device 100 may perform the in-communication-interruption control at the timing of starting step S11 in the information transmission control.

In the embodiment above, the control device 100 may omit the in-communication-interruption control. The control device 100 may thus omit the third transmission process. In addition, in response to this, the server 200 may omit the decision process. In this case, it is possible for the medical worker using the display 300 to comprehend the presence of the blank period during which the decision regarding the presence or absence of the respiratory therapy is not made.

The above embodiment illustrates the CPAP device program P for implementing the information transmission control and the in-communication-interruption control as a single program, but this is only an example. For example, a program for implementing the information transmission control and a program for implementing the in-communication-interruption control may be provided as separate packages. The program for implementing the information transmission control may also be provided as a plurality of packages. In this point, the same holds true for the program for implementing the in-communication-interruption control.

### APPENDIX

The following describe technical ideas derivable from the embodiment and the modifications above.

[1] A CPAP system includes: a CPAP device; and a reception part configured to communicate with the CPAP device. In the CPAP system, the CPAP device is configured to execute a determination process in which a determination about whether a respiratory therapy with the CPAP device has been performed within a predetermined fixed period is made, a first transmission process in which execution completion information is transmitted to the reception part in response to a determination in the determination process, the determination indicating that the respiratory therapy has been performed within the fixed period, the execution completion information indicating that the respiratory therapy has been performed within the fixed period, a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period. The reception part is configured to execute a surmise process in which a non-therapy period in which the respiratory therapy has not been performed is surmised based on the execution completion information and the non-execution information.

[2] In the CPAP system according to [1], the CPAP device is configured to execute a storing process in which therapy data during the respiratory therapy is stored in response to the respiratory therapy being performed, and transmits the therapy data to the reception part in the first transmission process, the therapy data being transmitted as the execution completion information or in addition to the execution completion information, the therapy data regarding the respiratory therapy performed within a period in which the respiratory therapy has been performed and that is indicated by the execution completion information.

[3] In the CPAP system according to [1] or [2], the reception part is configured to execute a reporting process in which information regarding the non-therapy period surmised in the surmise process is reported to an external device different from the CPAP device.

[4] In the CPAP system according to any one of [1] to [3], the reception part, in response to receiving of both of the execution completion information and the non-execution information failing in a given period in the surmise process, does not make a decision for a blank period regarding whether the respiratory therapy has been performed, the blank period being the given period.

[5] In the CPAP system according to [4], the CPAP device is configured to, in response to communication with the reception part being interrupted, and after the communication with the reception part becomes possible, execute a third transmission process in which the execution completion information and the non-execution information are transmitted to the reception part. The reception part is configured to execute a decision process in which a decision of whether the respiratory therapy has been performed within the blank period is made, the decision being made based on the execution completion information and the non-execution information that are transmitted in the third transmission process.

[6] A CPAP device is configured to execute: a determination process in which a determination about whether a respiratory therapy has been performed within a predetermined fixed period is made; a first transmission process in which execution completion information is transmitted to the reception part, the execution completion information indicating that the respiratory therapy has been performed within the fixed period, the execution completion information being transmitted in response to a determination in the determination process, the determination indicating that the respiratory therapy has been performed within the fixed period, and a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period.

[7] A CPAP device program is applied to a CPAP device including a control device and causes the control device to execute: a determination process in which a determination about whether a respiratory therapy has been performed within a predetermined fixed period is made, a first transmission process in which execution completion information is transmitted to a reception part in response to a determination in the determination process, the determination indicating that the respiratory therapy has been performed within the fixed period, the execution completion information indicating that the respiratory therapy has been performed within the fixed period, and a second transmission process in which non-execution information is transmitted to the reception part in response to a determination in the determination process, the determination indicating that the respiratory therapy has not been performed within the fixed period, the non-execution information indicating that the respiratory therapy has not been performed within the fixed period.

What is claimed is:

1. A continuous positive airway pressure (CPAP) system comprising:
a CPAP device; and
a receiver configured to communicate with the CPAP device,
wherein the CPAP device is configured to:
determine whether a respiratory therapy with the CPAP device has been performed within a predetermined fixed period,
transmit execution completion information to the receiver in response to determining that the respiratory therapy has been performed within the fixed period, the execution completion information indicating that the respiratory therapy has been performed within the fixed period,
transmit non-execution information to the receiver in response to determining that the respiratory therapy has not been performed within the fixed period, the non-execution information indicating that the respiratory therapy has not been performed within the fixed period, and
wherein the receiver is configured to surmise a non-therapy period in which the respiratory therapy has not been performed based on the execution completion information and the non-execution information.

2. The CPAP system according to claim 1, wherein the CPAP device is further configured to:
store therapy data during the respiratory therapy in response to the respiratory therapy being performed, and
transmit the therapy data to the receiver, the therapy data being transmitted as the execution completion information or in addition to the execution completion information, the therapy data regarding the performed respiratory therapy indicated by the execution completion information.

3. The CPAP system according to claim 1, wherein the receiver is further configured to report information regarding the surmised non-therapy period to an external device different from the CPAP device.

4. The CPAP system according to claim 1, wherein the receiver, in response to failing to receive both the execution completion information and the non-execution information in a given period, is configured to not determine whether the respiratory therapy has been performed, the given period being a blank period.

5. The CPAP system according to claim 4,
wherein the CPAP device is configured to, in response to communication with the receiver being interrupted, and after the communication with the receiver becomes possible, transmit the execution completion information and the non-execution information to the receiver, and
wherein the receiver is configured to determine whether the respiratory therapy has been performed within the blank period based on the transmitted execution completion information and the non-execution information after the communication with the receiver becomes possible.

6. A continuous positive airway pressure (CPAP) device configured to:
determine whether a respiratory therapy has been performed within a predetermined fixed period;
transmit execution completion information to a receiver in response to determining that the respiratory therapy has been performed within the fixed period, the execution completion information indicating that the respiratory therapy has been performed within the fixed period, and
transmit non-execution information to the receiver in response to determining that the respiratory therapy has not been performed within the fixed period, the non-execution information indicating that the respiratory therapy has not been performed within the fixed period.

7. A non-transitory computer-readable medium having instructions stored thereon that, when executed by a controller of a continuous positive airway pressure (CPAP) device, causes the CPAP device to:
determine whether a respiratory therapy has been performed within a predetermined fixed period,
transmit execution completion information to a receiver in response to determining that the respiratory therapy has been performed within the fixed period, the execution completion information indicating that the respiratory therapy has been performed within the fixed period, and
transmit non-execution information to the receiver in response to determining that the respiratory therapy has not been performed within the fixed period, the non-execution information indicating that the respiratory therapy has not been performed within the fixed period.
